(19) United States
(12) Plant Patent Application Publication
(10) Pub. No.: US 2016/0338234 P1
(43) Pub. Date: Nov. 17, 2016

(54) Vaccinium plant named 'JAAC'

(50) ;

(71) Applicants: Lancelot Alan Jordan, Hamilton (NZ); Jeanette Jordan, Hamilton (NZ)

(72) Inventors: Lancelot Alan Jordan, Hamilton (NZ); Jeanette Jordan, Hamilton (NZ)

(21) Appl. No.: 14/545,511

(22) Filed: May 14, 2015

(51) Int. Cl. A01H 5/00

(52) U.S. Cl. PLT/157

(57) ABSTRACT

A blueberry plant particularly distinguished by large oblate fruit with sweet flavour, crisp texture, strong blue colour and abundant bloom; rounded leaves with a puckered surface texture; upright plant habit with good vigour and green appearance and deciduous foliage, is described.

### BACKGROUND OF THE INVENTION

The present invention relates to a new and distinct variety of Vaccinium corymbosum (blueberry) hereinafter referred to by the cultivar name ‘JAAC’ or the ‘new plant’. ‘JAAC’ was discovered as a plant of unknown parentage in a population of ‘Sunset Blue’ (U.S. Plant Patent No. 21,761) in February 2014 in Gordonton, Waikato, New Zealand.

The new plant has been asexually propagated by softwood cuttings in Cambridge, Waikato, New Zealand and the unique combination of characteristics of the new plant has been found to be reproducible and stable in propagation.

Plant Variety rights application for ‘JAAC’ was filed in New Zealand on 20 March, 2015 and assigned the application number BLU054. Propagation material of ‘JAAC’ has never been sold or made publically available prior to the filing of this application.

### SUMMARY OF THE INVENTION

The following are the most outstanding and distinguishing characteristics of the new plant when grown under normal horticultural practices in Gordonton, Waikato, New Zealand:

1. Large oblate fruit with sweet flavour, crisp texture, strong blue colour and abundant bloom

2. Rounded leaves with a puckered surface texture

3. Upright plant habit with good vigour and green appearance

4. Deciduous foliage

### DESCRIPTION OF THE PHOTOGRAPHS

The new plant is illustrated by the accompanying photographs which show in full color typical specimens of the plant habit, foliage, fruit and flowers of the new variety ‘JAAC’.

The colors shown are as true as can be reasonably obtained by conventional photographic procedures. The photographs include plants approximately five years' old grown in optimum conditions in Gordonton, Waikato, New Zealand

FIG. 1 shows the plant habit

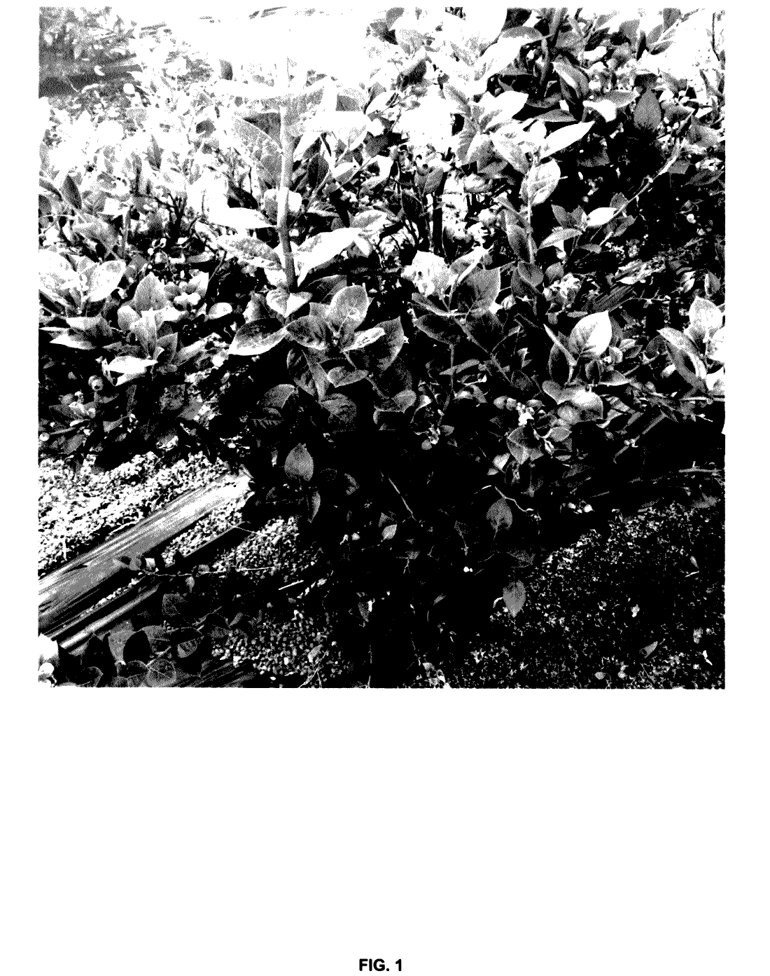

FIG. 2 shows a close-up of a mature leaf upper side

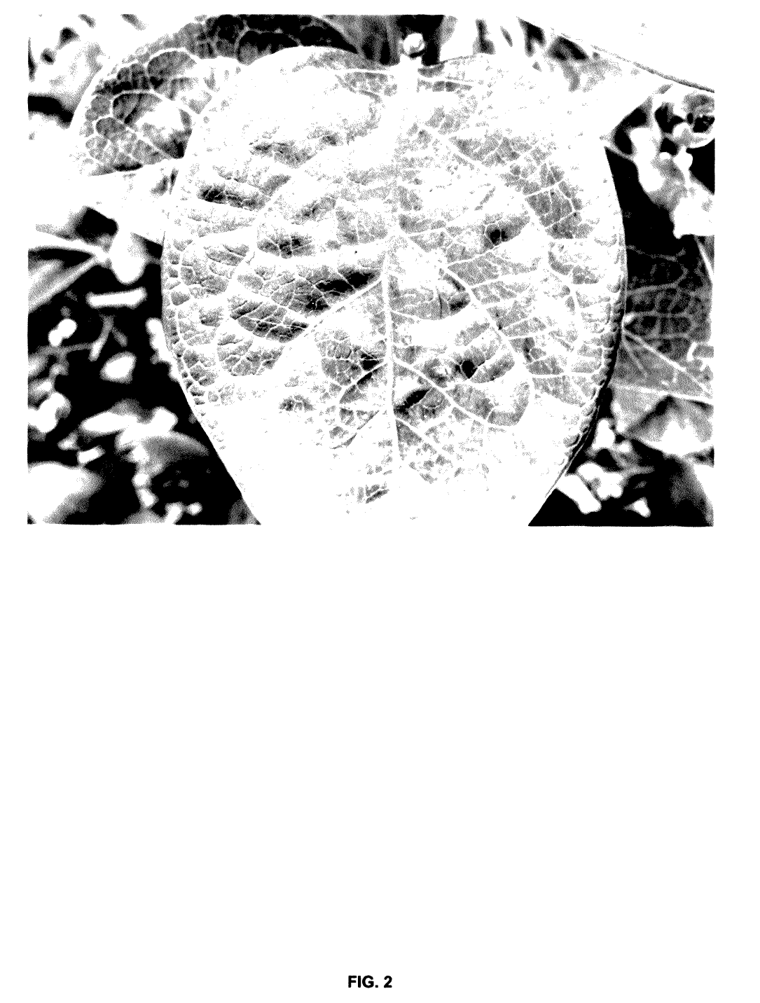

FIG. 3 shows mature leaves upper and under side and one year wood

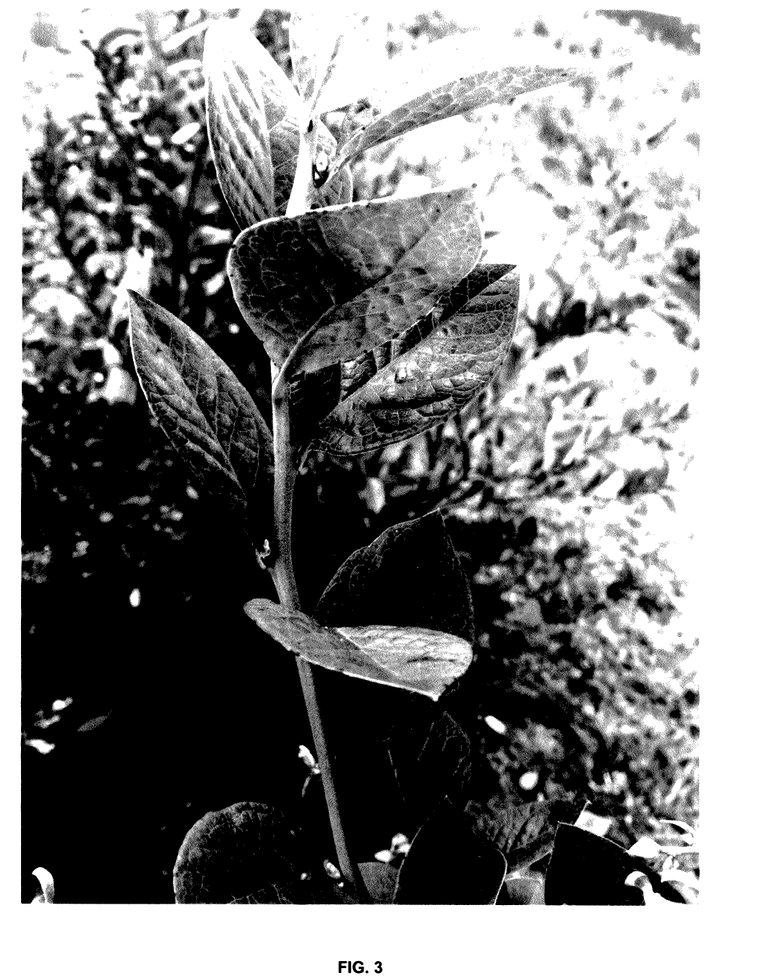

FIG. 4 shows an open flower and flower buds

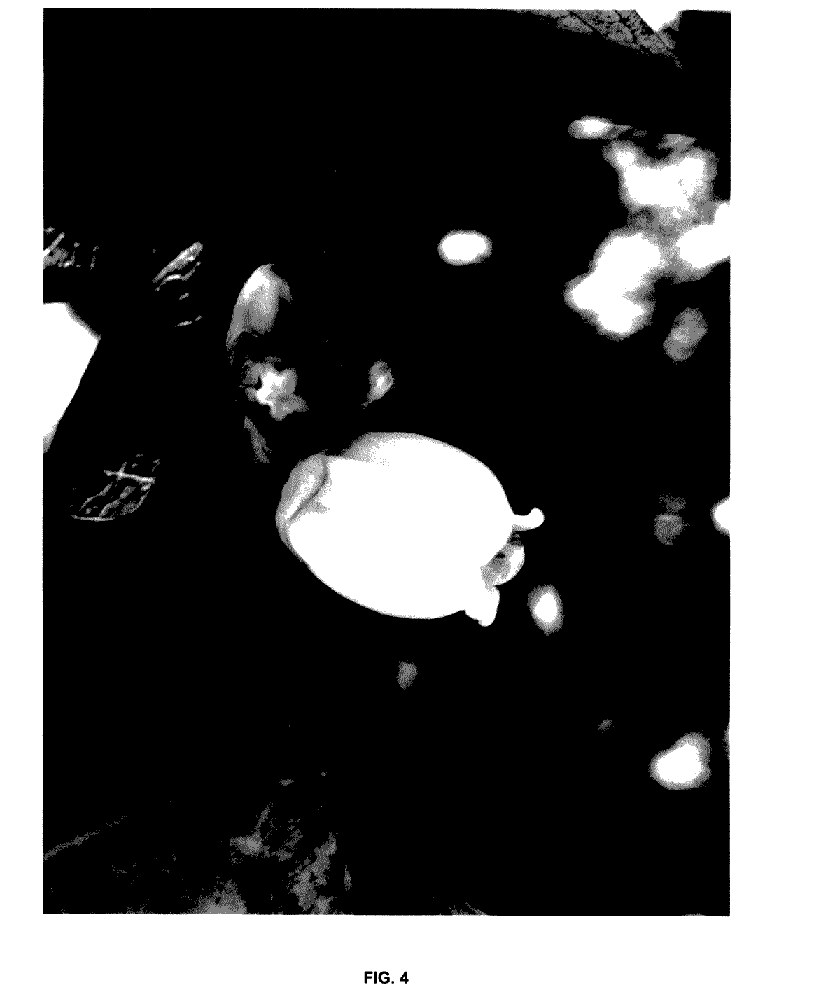

FIG. 5 shows unripe and ripe berries

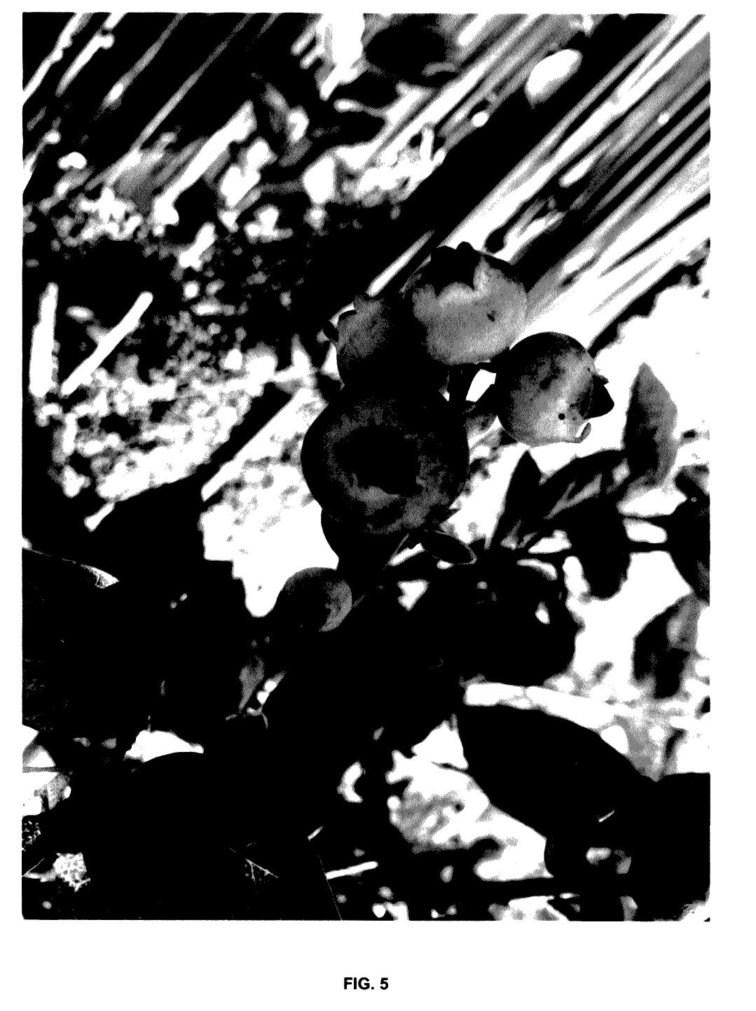

FIG. 6 shows ripe berries

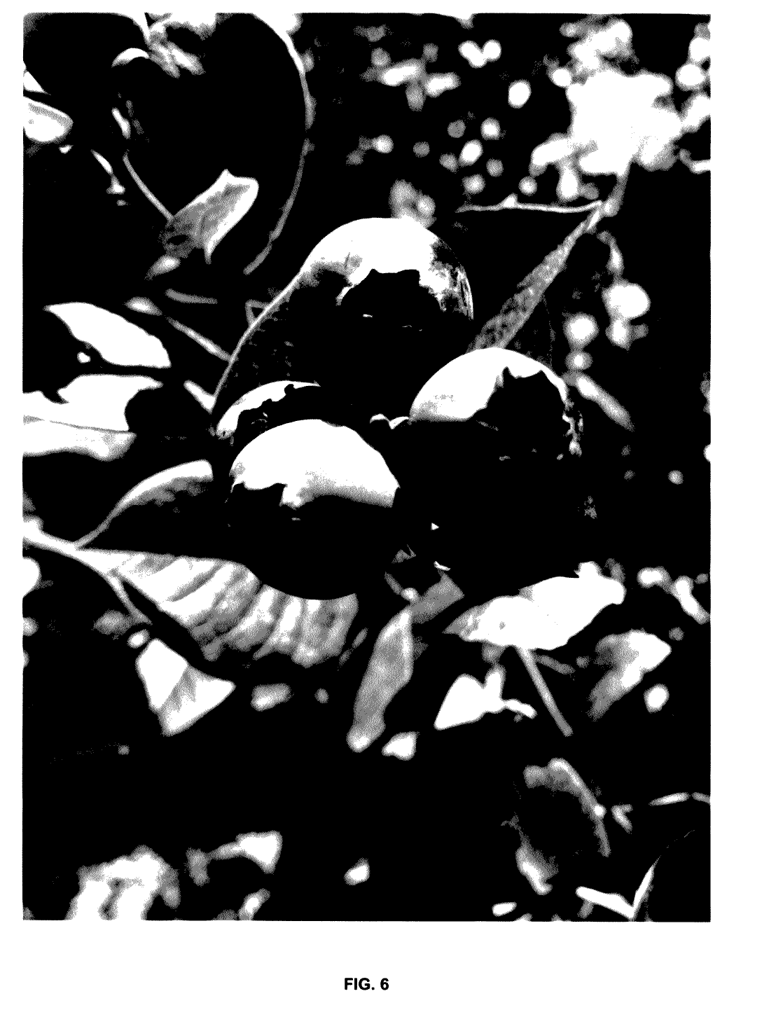

FIG. 7 shows berry flesh and seeds from a ripe berry

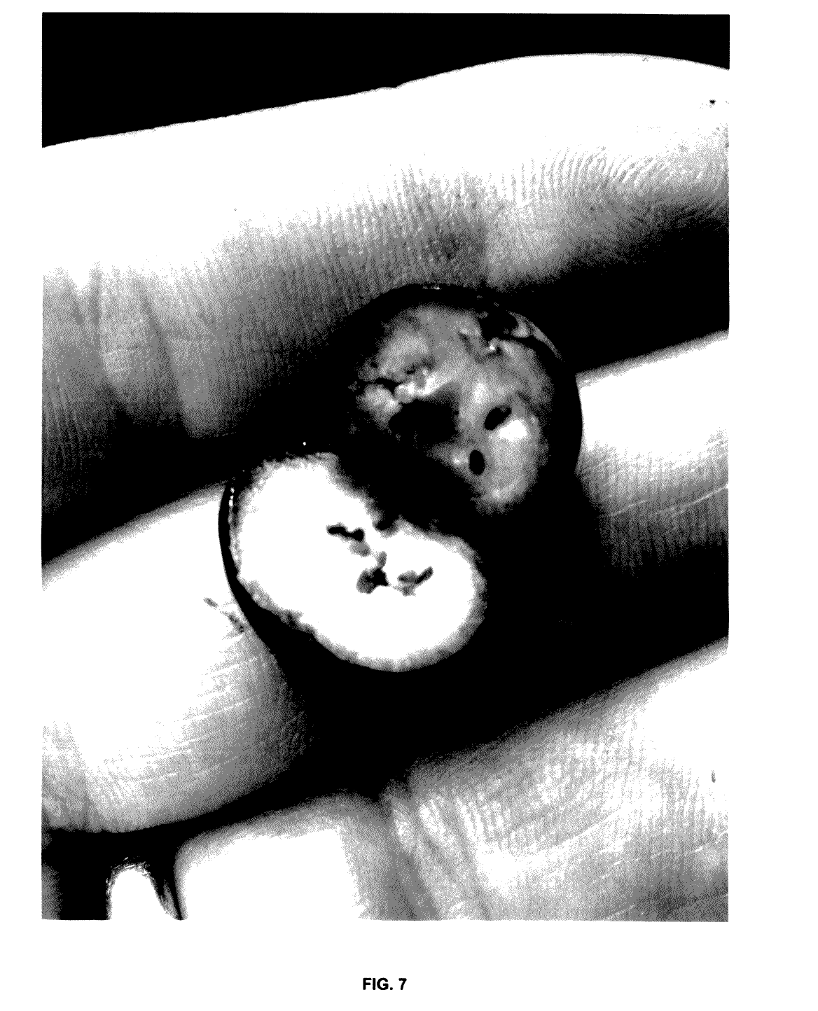

### DESCRIPTION OF THE NEW CULTIVAR

The following detailed description sets forth the distinctive characteristics of ‘JAAC’. The following observations, measurements and values were made on five year old plants, and managed under standard orchard practice. Random measurements of each characteristic were obtained from samples of two plants unless otherwise stated.

Horticultural terminology is used in accordance with UPOV guidelines for Vaccinium. All dimensions are in millimetres, and all weights are in grams (unless otherwise stated). Certain characteristics of this variety, such as growth and color, may change with changing environmental conditions (e.g., light, temperature and moisture), nutrient availability, or other factors. Color readings were taken in natural light. Color references are primarily to the 1995 Edition of the Colour Chart published by the Royal Horticultural Society, London (RHS), except where general colours of ordinary significance are used.

### Detailed Botanical Description

Classification:

Family: Ericaceae

Botanical name: Vaccinium corymbosum

Common name: blueberry

Parentage:

Female parent: unknown

Male parent: unknown

Plant:

Plant habit: upright plant with erect multiple canes, good vigour

Plant Height: average 120 cm

Plant Width: average 100 cm

Propagation: softwood cuttings

Branches:

Texture of new wood: smooth with inconspicuous lenticels

Colour of new wood: yellow-green, RHS 144B with red-purple anthocyanin colouration RHS 60B

Texture of one year wood: smooth with inconspicuous lenticels

Colour of one year wood: RHS 144B with red-purple anthocyanin colouration RHS 60B

Texture of three year wood: rough and barky

Colour of three year wood: grey-green, RHS 197D

Diameter of three year wood: 10 mm to 20 mm

Leaves:

Arrangement: alternate and decussate

Length (young leaf): 44 mm to 54 mm

Width (young leaf): 30 mm to 35 mm

Length (mature leaf): 55 mm to 80 mm

Width (mature leaf): 32 mm to 55 mm

Shape: broadly ovate

Apex: broadly acute

Base: attenuate

Margin: entire

Texture:
Upper surface: matt, leathery with raised puckering
Lower surface: matt, smooth

Colour:
Young leaves, upper surface: green, RHS 137A
Young leaves, lower surface: green, RHS 138B
Mature leaves, upper surface: green, RHS 137A with red-purple anthocyanin colouration RHS 60B
Mature leaves, lower surface: green, RHS 138B with red-purple anthocyanin colouration RHS 60B

Venation:
Pattern: reticulate
Colour, upper surface: RHS 139C with red-purple anthocyanin colouration RHS 60A on main vein and RHS 60D on lateral veins
Colour, lower surface: RHS 139D with red-purple anthocyanin colouration RHS 60A on main vein and RHS 60D on lateral veins

Petiole (leaf stem):
Length: 3 mm to 7 mm
Diameter (cross section) 1 mm to 2 mm
Colour: yellow-green, RHS 144B with red-purple anthocyanin colouration RHS 60B
Texture: smooth

Inflorescence

Fragrance: lightly scented in the early morning

Longevity of viable flowers on the plant: 14 days

Peduncle (stalk holding each flower cluster):
Length: 1 mm to 2 mm
Diameter: 2 mm to 4 mm
Colour: yellow-green, RHS 144B with red-purple anthocyanin colouration RHS 60B

Pedicel (stalk holding each flower):
Length: 4 mm to 5 mm
Diameter: 2 mm to 4 mm
Colour: yellow-green, RHS 144B with red-purple anthocyanin colouration RHS 60B

Flowers:
Number of bud clusters per fruiting (terminal) shoot: 5-12
Flower number per cluster: 4-7
Colour of flower bud: white, RHS 155B with occasional red-purple anthocyanin colouration RHS 58B on outside near calyx
Shape of flower: urceolate
Colour of corolla (fully opened): white RHS 155B
Length of corolla tube: 9 mm to 11 mm
Width of corolla tube: 7.5 mm to 8 mm
Width of corolla tube aperture: 4 mm to 5 mm

Calyx:
Quantity of lobes: 5
Length of lobes: 4 mm (average)
Width of lobes: 3.5 mm (average)
Apex of lobes: broadly acute
Margin: smooth
Texture: smooth
Colour: yellow-green, RHS 144B with occasional red-purple anthocyanin colouration RHS 60B on margin

Reproductive Organs:

Style:
Quantity: 1
Length: 10 mm to 12 mm
Attitude: erect
Colour: yellow-green, RHS 144B

Stamens:
Quantity: 10

Filaments:
Colour: yellow-green, RHS 144D
Texture: lightly pubescent

Anthers:
Colour: greyed-orange, RHS 167A

Ovary:
Shape: round
Colour: yellow-green, RHS 144D

Ovules:
Colour: yellow-green, RHS 144D with dark brown internal markings

Fruit:

Yield: high, average 3 kg per 5 year old bush

Colour: Colour of unripe berry: red-purple RHS 60B
Ripe berry colour with bloom: blue, RHS 103A. No red colouration around stem end of ripe berry.
Ripe berry colour without bloom: black, 202A
Abundance of surface bloom: high
Berry flesh colour: green-yellow, RHS 10

Calyx: Calyx width: 6 mm to 8 mm
Calyx depth: 2 mm to 3 mm
Calyx shape: five lobed

Berry shape: oblate

Berry weight range: 2.5 g to 3 g

Average berry weight: 2.5 g

Berry diameter: 16 mm (average)

Berry depth: 11 mm (average)

Aspect (WW): 1.45

Cluster density: open

Detachment force: medium-firm

Pedicel (fruit stem) scar size: average 2.5 mm

Pedicel (fruit stem) scar dryness: dry

Berry firmness:

Flavour:
Taste: sweet flavourful
Texture: smooth, very little grit, crisp to bite
Brix level at consumption: 16% average

Suitability for mechanical harvest: not considered

Seed:

Seed abundance: 20 to 25 per berry

Seed colour: greyed-orange, RHS 166B

Seed size: very small 1 mm average

Crop Timing:

Vegetative bud burst: October to November

Beginning of flowering: September

Beginning of fruit ripening: late January

Fruit harvest period: 1 February to 1 May

Leaf Senescence: late season deciduous

Disease Resistance:
No observations made.

Insect Resistance:
No observations made.

### Comparison with Parental and Known Cultivars

When compared to the commercial variety ‘Sunset Blue’ (U.S. Plant Patent number 21,761); ‘JAAC’ has a more upright plant habit, is later fruiting and has later leaf senescence.

When compared to the commercial variety ‘O'Neal’ (unpatented); ‘MAC’ has a broader leaf, a puckered upper leaf texture and a larger average berry size.

When compared to the commercial variety ‘Maru’ (unpatented); ‘JAAC’ has more vigorous growth, denser branches, is deciduous, and berries are oblate, larger with thinner skin and smoother texture.

I claim:

1. A new and distinct cultivar of blueberry plant as shown and described herein.
